(19) United States
(12) Patent Application Publication
(10) Pub. No.: US 2003/0177509 A1
(43) Pub. Date: Sep. 18, 2003

(54) Mouse model for HIV infection utilizing a chimeric HIV/MLV Gag protein that permits efficient assembly from murine cells

(75) Inventors: Benjamin K. Chen, Brookline, MA (US); Peter S. Kim, Bryn Mawr, PA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Whitehead Institute for Biomedical Research, Cambridge, MA

(21) Appl. No.: 10/236,064

(22) Filed: Sep. 4, 2002

Related U.S. Application Data
(60) Provisional application No. 60/316,999, filed on Sep. 4, 2001.
(60) Provisional application No. 60/340,619, filed on Dec. 10, 2001.

(51) Int. Cl. A01K 67/027; C12Q 1/70; C12N 7/00; C12N 5/06

(52) U.S. Cl. 800/11; 800/18; 435/5; 435/235.1; 435/354

(57) ABSTRACT

Functional chimeric proteins that comprise HIV Gag protein and a Gag protein of non HIV origin (e.g., of viral or retroviral origin other than HIV origin) and overcome the block to HIV assembly in non human cells. One embodiment of the present invention is chimeras between HIV Gag protein and a Gag protein from a murine virus, such as Moloney Murine Leukemia Virus (MLV) Gag protein, that overcome the block to HIV assembly in mouse cells.

### RELATED APPLICATIONS

This application claims the benefit of the filing date of U.S. Provisional Application No. 60/316,999, filed Sep. 4, 2001 and entitled Mouse Model for HIV Infection Utilizing a Chimeric HIV/MLV Gag Protein That Permits Efficient Assembly From Murine Cells, by Benjamin K. Chen and Peter S. Kim and U.S. Provisional Application No. 60/340,619, filed Dec. 10, 2001 and entitled Mouse Model for HIV Infection Utilizing a Chimeric HIV/MLV Gag Protein That Permits Efficient Assembly From Murine Cells, by Benjamin K. Chen and Peter S. Kim The teachings of these referenced applications are expressly incorporated herein by reference.

### GOVERNMENT FUNDING

This invention was made with government support under National Research Service Award 1 F32 A110664-01 awarded by the National Institutes of Health/NIAID and under Grant Number GM44162 awarded by the National Institutes of Health. The government has certain rights in the invention.

### BACKGROUND OF THE INVENTION

HIV researchers have long sought a genetically tractable alternative to primate models for HIV infection. A small animal model for HIV infection would provide an alternative for testing of vaccines or therapeutics and could also further understanding of the pathogenesis of disease

### SUMMARY OF THE INVENTION

The present invention relates to functional chimeras comprising HIV Gag protein and Gag protein of non HIV origin (e.g., of viral origin or retroviral origin other than HIV origin) that overcome the block to HIV assembly in non human cells. The functional chimeras can additionally comprise further components, as described herein The functional chimeras can comprise a.) complete HIV Gag protein or less than the complete HIV Gag protein and b.) complete Gag protein of non HIV origin or less than a complete Gag protein of non HIV origin. The functional chimeras can comprise, for example, HIV Gag protein and a Gag protein from a murine virus, such as Moloney Murine Leukemia Virus (MLV) Gag protein. A functional chimera can comprise. for example, complete HIV Gag protein and a portion of MLV Gag protein such as MLV Gag matrix (MA) protein, or MLV Gag MA protein and MLV p12 protein Alternatively, functional chimeras of the present invention comprise less than a complete HIV Gag protein and Gag protein of non HIV origin or a portion of a Gag protein of non HIV origin. In specific embodiments, functional chimeras of the present invention comprise a.) HIV Gag protein that lacks the HIV Gag MA domain or a portion of the MA domain and b.) all or a portion of the MA domain of a Gag protein of non HIV origin, such as the MA domain of MLV. In these embodimdents, the MA domain(s) or MA domain portions present must be of sufficient composition (amino acid residues) to overcome the block to HIV assembly that occurs in non human cells. For example, a functional chimera can comprise a.) HIV Gag protein that lacks the HIV Gag MA domain and b.) a complete MA domain of non HIV origin, such as the MA domain of MLV. Alternatively, a functional chimera can comprise a.) HIV Gag protein that includes a partial HIV Gag MA domain and b.) a complete MA domain of non HIV origin, such as the MA domain of MLV. In additional embodiments, the functional chimeras can comprise a.) HIV Gag protein that lacks the HIV Gag MA domain and b.) a portion of a MA domain of non HIV origin, such as the MA domain of MLV or can comprise a.) HIV Gag protein that includes a partial EMV Gag MA domain and b.) a complete MA domain of non HIV origin, such as the MA domain of MLV. In specific embodiments of the present invention, HIV p17 MA is replaced with p15 of MLV Gag.

In further embodiments, the functional chimeras additionally comprise MLV p12 protein or a sufficient portion to overcome the block to HIV assembly in non human cells. In these embodiments, for example, a functional chimera can comprise a.) HIV Gag protein that lacks the HIV Gag MA domain and b.) a complete MA domain of non HIV origin, such as the MA domain of MLV, and MLV p12 protein or a sufficient portion to overcome the block to HIV assembly in non human cells. Alternatively, a functional chimera can comprise a.) HIV Gag protein that includes a partial HIV Gag MA domain and b.) a complete MA domain of non HIV origin, such as the MA domain of MLV and MLV p12 protein or a sufficient portion to overcome the block to HIV assembly in non human cells. In additional embodiments, the functional chimeras can comprise a.) HIV Gag protein that lacks the HIV Gag MA domain and b.) a portion of a MA domain of non HIV origin, such as the MA domain of MLV or can comprise a.) HIV Gag protein that includes a partial HIV Gag MA domain and b.) a complete MA domain of non HIV origin, such as the MA domain of MLV, and MLV p12 protein or a sufficient portion to overcome the block to HIV assembly in non human cells. In specific embodiments, HIV p17 MA is replaced with the p15 MA domain and the p12 domain of MLV Gag. Functional HIV Gag protein-MLV Gag protein chimeras are referred to herein as functional HIV-MLV Gag chimeras; functional chimeric HIV particles and functional HIV Gag protein-MLV Gag protein chimeras.

Further embodiments of the present invention are nucleic acids (DNAs, RNAs) that encode such functional chimeras (e.g., DNAs or RNAS that encode functional HIV-MLV Gag chimeras); mouse cells that contain chimeras of the present invention, particularly functional HIV-MLV Gag chimeras; mouse cells that express DNA encoding such chimeras, particularly HIV-MLV Gag chimeras; and mouse models that express functional chimeras that overcome the block to HIV assembly in their cells (mouse models in which HIV assembly occurs in cells). DNAs and RNAs of the present invention can be produced by known methods, such as recombinant nucleic acid (DNA, RNA) technology or chemical synthetic methods. The present invention further relates to methods of screening for therapeutic agents, in which mice of the present invention or cells obtained from such mice and maintained in culture are used; methods of testing for or identifying vaccines that prevent or reduce HIV infection in which mice of the present invention are used and methods of aiding in the identification of such vaccines (e.g., by assessing whether a vaccine candidate identified by another means provides protection in vivo).

The chimeras, mouse model and methods of the present invention can be used to provide an animal model for identifying, developing and testing vaccines and therapeutic agents useful in preventing and treating HIV infection, as well as to provide a model valuable for determining host genetics of HIV infection.

### BRIEF DESCRIPTION OF THE DRAWINGS

FIG. 1 is a schematic illustration of the MLV/HIV Gag Chimeras. Genomic organization of HIV (top) with details of the Gag structure and chimeric strategies are shown below. HIV open reading frames are represented by open rectangles. MLV open reading frames are shaded rectangles.

FIGS. 2A and 2B are graphic representations of levels of p24 antigen harvested from supernatants from two cell types and demonstrate that chimeric MA12-HIV releases greater levels of supernatant p24 in transfected murine cells. FIG. 2A is a graph that represents levels of p24 antigen harvested from supernatants from transfected human embryonic Kidney cells 293. FIG. 2B is a graph that represents levels of p24 antigen harvested from supernatants from transfected 3T3 XTC cells. p24 ELISA was performed on 0.45 micron filtered supernatants prepared from transfected cells. Results are indicative of 3 independent experiments.

FIG. 3 is a graphic representation of results that show that MHIV MA12 Luc virus is able to undergo single round infection when produced from human cells and provided with an HIV Env with a truncated cytoplasmic tail ( [symbol] 147 Env) but not wild type Env (WT Env). Virus containing supernatants were prepared from transfected 293 cells following cotransfection with an envelope-deficient, luciferase encoding, provirus (HIV Luc, MHIV MA Luc, or MHIV MA12 Luc) and an envelope expression vector (SRHS Env or SRHS [symbol] 147 Env). Infectivity of the viruses was measured by luciferase activity from cell lysates prepared from infected HOST4 (CD4+) cells at 48 hours postinfection.

FIG. 4 is a schematic representation of the original MHIV MA chimeras version 2 (v2) chimeras and dual matrix MHIV Gag chimeras. Genomic organization of HIV-1 is shown in the top panel. The HIV-1 Gag polyprotein is processed into p17 MA, p24, CA, p2, p7 NC, p1 and p6. Chimeric strategies replace the p17 MA with p15 MA of MLV Gag (MHIV MA) or both the p15 MA and p12 domains of MLV Gag (MHIV MA 12). V2 chimeras (MHIV MA v2 and MHIV MA12 v2) carry the HIV MA-CA protease cleavage site at the end of the inserted MLV MA sequence. Dual matrix chimeras replace the HIV p17 MA with p15 MA of MLV Gag followed by p17 MA of HIV Gag (DMA-1) or p17 MA of HIV Gag followed by p15 MA of MLV Gag (DMA-2). DMA constructs carry the HIV MA-CA protease cleavage sites at all new subunit junctions. For reference, the complete MLV Gag consists of p15 MA, p12, p30 CA and p10 NC, bottom panel. Scale of viral genome sequence is shown in nucleotides (nt).

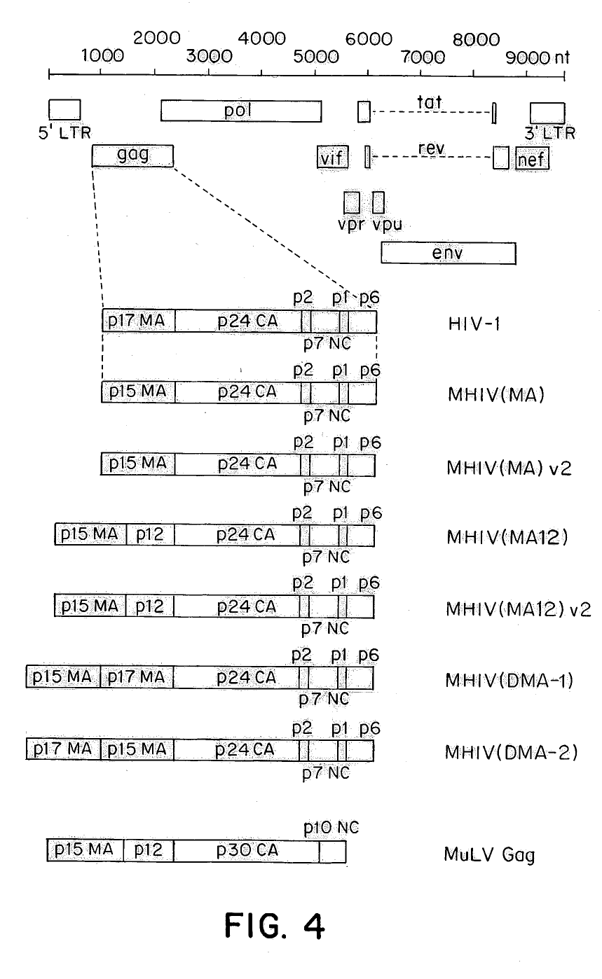

FIG. 5 is a schematic representation of the MHIV cytoplasmic tail ENV chimeras. Genomic organization of HIV-1 is shown in the top panel. Env structure consists of two subunits: surface glycoprotein; gp120 and transmembrane gp41. Transmembrane (TM) and cytoplasmic (CT) regions in gp41 are indicated. Non-chimeric ΔCT construct carries a deletion of the HIV CT. Chimeric Env constructs fuse the cytoplasmic tail af MLV Env onto the TM and ectodomain of HIV Env (MCT1). A second Env chimera replaces the entire transmembrane domain TM and the CT portions of HIV Env with a comparable sequence from MLV Env (MCT2). The structure of the complete MLV Env is shown below consisting of gp70 and p12E. Scale of viral genome sequence is shown in nucleotides (nt).

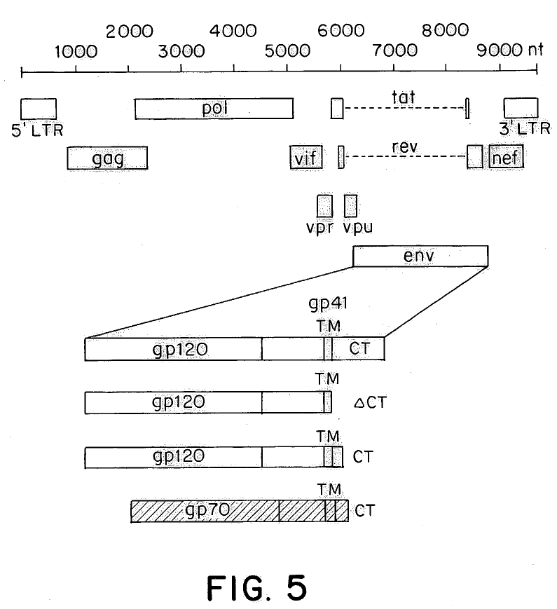

### DETAILED DESCRIPTION OF THE INVENTION

Murine cells are nonpermissive for human immunodeficiency virus infection due to blocks in entry, gene expression and assembly. The block to entry can be overcome effectively by the addition of human CD4 and CCR5 or CXCR4. Low levels of HIV gene expression in mouse cells can be significantly enhanced by the addition of the Tat cofactor, CyclinT1.

However, even when high levels of HIV proteins are made in mouse cells, they fail to assemble efficiently into virus particles. The HIV Gag protein is the only viral protein required to assemble HIV particles from human cells. The N-terminal domain of HIV Gag, matrix (MA), targets Gag to the plasma membrane, where assembly occurs. The inability of mouse cells to support HIV assembly is thought to occur because the HIV Gag protein is mislocalized or mistargeted in mouse cells and found in intracellular aggregates. To determine what portion of HIV Gag is incompatible with the mouse cellular machinery, Applicants have made functional chimeras between HIV and the Moloney Murine Leukemia Virus (MLV) Gag. Chimeras which replace the HIV MA with the MLV MA or MLV MA and p12 were examined in human 293 cells and murine NIH 3T3 fibroblasts expressing CD4, CXCR4 and CyclinT1. The MA-p12 chimera released levels of p24 similar to normal HIV when transfected into human 293 cells) but produced 15-fold greater release of p24 when expressed in murine cells. Deconvolution fluorescence microscopy of transfected murine cells revealed that the chimeric Gag protein is membrane localized with a punctate pattern, while the HIV Gag protein shows no membrane localization. Western blot analysis of the chimeric particles shows that the Gag polyprotein is efficiently processed by the HIV protease, but the levels of HIV Env are very low. When pseudotyped with an HIV env containing a truncated cytoplasmic tail, the chimeric virus is infectious in a single round. These results provide evidence that it is specifically the MA domain of HIV Gag that is incompatible with assembly in mouse 3T3 cells.

Since the discovery of CD4 as the cell receptor for HIV, it has been known that the addition of CD4 was insufficient to allow HIV replication in mouse cells (10). This observation led to the co-receptor hypothesis—that an additional species-specific factor was required for HIV entry. The co-receptor hypothesis motivated functional cloning experiments that used murine cells to identify a chemokine receptor, CXCR4, as a co-receptor essential for HIV membrane fusion (7). Mouse cells were also known to have a defect in supporting HIV Tat, a protein needed for efficient viral gene expression. Experiments with human-rodent somatic cell hybrids revealed that a gene on human chromosome 12 could restore Tat function (8, 14). Following the biochemical purification of CyclinT1 as a key Tat cofactor (17), it was revealed to be the Tat co-factor that was missing from mouse cells.

Investigators then tested whether HIV could infect mouse cells stably expressing CD4, CXCR4 or CCR5, and Cyclin T1. HIV could enter these cells. The addition of the human CyclinT1 to mouse cells restored high levels of HIV gene expression, but HIV still would not replicate in these cells (12). In CyclinT1-expressing mouse cells, high levels of Gag were made following infection, however, the Gag protein was mistargeted and virus assembly was extremely inefficient. Electron micrographs showed intracellular electron dense intracellular structures that appeared to be aggregates of HIV Gag protein (12). This block to viral assembly was relieved by fusion of the infected mouse cells to permissive human cells (2, 11), illustrating a specific requirement for a human cellular factor(s) in particle assembly.

The N terminal domains of retroviruses are frequently referred to as matrix domains (MA) and are responsible for the proper localization of the Gag polyprotein in cells. MA proteins are almost invariably N-myristoylated. This lipid modification, along with a cluster of basic amino acids, is responsible for membrane localization of the Gag protein. HIV MA targets the Gag protein specifically to the plasma membrane. Gag proteins with mutations in the MA domain have been described that direct virus assembly to other membrane compartments (15).

Until the work described herein, it was unclear what portion of the HIV genome is incompatible with assembly in mouse cells. In view of the fact that the phenotype in mouse cells appears to be one of misguided localization, it is possible that signals present in HIV MA are unrecognized by the mouse machinery. To address test this possibility, Applicants replaced HIV MA with N-terminal sequences from the murine leukemia virus (MLV) Gag protein. MLV is a murine retrovirus that normally functions in mouse cells. The aim was to make functional, chimeric HIV Gag protein that allows virus assembly in mouse cells. Applicants have identified a chimeric Gag protein that localizes properly and directs assembly more efficiently than the normal HIV Gag in murine 3T3 cells. When produced by high efficiency transfection of human 293 cells, this virus also retains its ability to infect cells in a single round. It is reasonable to expect that a MLV-HIV chimeric virus provides a viable option for use in a mouse model for HIV infection.

Thus, Applicants have produced functional chimeric HIV-1 Gag protein. That is, they have produced murine HIV (MHIV) expressing a chimeric Gag protein that localized properly and directed assembly more efficiently in murine 3T3 cells than the nonchimeric HIV-1 Gag.

The present invention relates to functional HIV-MLV Gag chimeras which comprise a.) HIV Gag protein or a portion thereof and b.) Gag protein of non HIV origin or a portion thereof, which overcomes the block to HIV assembled in non human cells. In specific embodiments, the Gag protein of non HIV origin is a murine virus Gag protein, such as the Gag protein of Moloney Murine Leukemia Virus. In further embodiments, the Gag protein of non HIV origin is MLV Gag matrix protein. Functional chimera of the present invention, in one embodiment, comprise HIV Gag protein or a portion thereof and MLV Virus Gag matrix protein or a portion thereof and MLV p12 protein or a portion thereof. In specific embodiments, MLV Matrix or portion thereof is positioned in the functional chimera at the N terminus or within the HIV Gag protein.

The present invention further relates to functional HIV-MLV Gag chimera that comprise, in addition to the components described above, HIV Env in which the cytoplasmic tail is truncated in such a manner that the functional chimera produces infectious particles in mouse cells. In these embodiments, the functional chimeras comprise a.) HIV Gag protein or a portion thereof; b.) Gag protein of non HIV origin or a portion thereof; and c.) HIV Env in which the cytoplasmic tail is truncated in such a manner that the functional chimera produces infectious particles in mouse cells. In these embodiments, the Gag protein of non HIV origin can be a murine virus Gag protein, such as the Gag protein of MLV. As in embodiments described above, te HIV Gag matrix domain can be replaced by the Gag matrix domain of a murine virus, such as MLV.

The present invention further relates to nucleic acid constructs (DNA or RNA) that encode a functional chimeric HIV particle (functional HIV Gag protein-MLV Gag protein chimera) that is assorted in non human cells, wherein the chimeric HIV particle comprises chimeric HIV Gag protein in which the HIV Gag matrix domain is replaced by the Gag matrix domain of a murine virus. Nucleic acid constructs that encode any of the functional HIV Gag protein-MLV Gag chimeras described herein are also the subject of the present invention. In specific embodiments, nucleic acid constructs of the present invention encode a chimeric HIV particle which comprises all or a portion of tne HIV Gag protein and a Gag protein of non HIV origin, such as a murine virus Gag protein (e.g., Moloney Murine Leukemia Virus). The encoded murine virus Gag protein can be, for example, all or a portion of MLV Gag matrix protein. In a further embodiment the nucleic acid construct encodes a functional chimeric HIV particle that comprises all or a portion of HIV Gag protein, all or a portion of the Gag matrix domain of a murine virus (e.g., all or a portion of MLV Gag matrix protein) and MLV p12 protein. In a specific example, the nucleic acid construct encodes a functional chimeric HIV particle that comprises HIV Gag protein that lacks all or a portion of the HIV matrix domain; all or a portion of the Gag matrix domain of MLV; and MLV p12 protein.

The present invention further relates to a method of identifying or screening for a drug or agent that inhibits HIV infection of cells; a method of assessing the ability of a candidate inhibitor of HIV infection to inhibit (partially or completely) HIV infection of cells; and nucleic acid constructs and mouse models useful in such methods. In one embodiment, the method of identifying or screening for a drug that inhibits HIV infection of cells is carried out as follows: A drug to be assessed for its ability to inhibit HIV infection of cells (a candidate drug) is administered to a transgenic mouse whose cells express human CD4, human CXCR4 or human CCR5 and human Cyclin T1 and a functional chimeric HIV Gag protein-MLV Gag protein; the mouse is maintained for sufficient time and under suitable conditions for infection of cells by the functional chimeric protein to occur and the extent to which infection occurs is determined. If infection of cells in mice to which the candidate drug is administered (test mice) is less (e.g., occurs to a lesser extent/in fewer cells in a given period of time) than in control mice, then the candidate drug is a drug that inhibits HIV infection of cells. Control mice are typically the transgenic mouse as described above who are treated in the same manner as the test mice except that the drug being assessed is not administered. The ability of a candidate inhibitor, such as a candidate inhibitor identified through an in vitro assay, a cell-based assay or other method (e.g., rational drug design based on computer modeling), to inhibit HIV infection of cells in an animal can also be assessed using a transgenic mouse whose cells express human CD4, human CXCR4 or human CCR5 and human Cyclin T1 and a functional chimeric HIV Gag protein-MLV Gag protein. The candidate inhibitor is administered to the transgenic mouse, the mouse is maintained for sufficient time and under suitable conditions for infection of cells by the functional chimeric protein to occur and the extent to which infection occurs is detemined. If infection of cells in mice to which the candidate inhibitor is administered (test mice) is less (e.g., occurs to a lesser extent/in fewer cells in a given period of time) than in control mice, then the candidate inhibitor is an inhibitor of HIV infection of cells.

Cells that express human CD4, human CXCR4 or human CCR5 and human Cyclin T1 can also be used to identify or screen for a drug or agent that inhibits HIV infection of cells. Such cells can be used in a method of the present invention as follows: Cells, referred to as test cells, are cultured in the presence of a nucleic acid construct encoding a functional chimeric HIV Gag protein-MLV Gag protein of the present invention and a drug to be assessed for its ability to inhibit HIV infection of cells (a candidate drug), under conditions appropriate for infection of the cells by the functional chimeric protein. Infection of test cells is assessed and compared with infection of control cells, which are the cells cultured as described above except that no drug to be assessed is present. If infection of cells is less in the presence of the candidate drug than in its absence, the candidate drug is able to inhibit HIV infection of cells (is an inhibitor of HIV infection of cells).

The present invention is illustrated by the following exemplification, which is not intended to be limiting in any way.

### EXEMPLIFICATION

The following methods and materials were used in the work described herein.

Materials and Method

Plasmids and cell lines. Proviral constructs were based upon the HIV molecular clone, pNL4-3 (1). MLV Gag sequences were derived from packaging vector, pCL Eco (13). Chimeric viruses were generated by two-step PCR strategy utilizing primers that join the exact MLV MA or MA/p12 sequence in place of the HIV MA domain. The 5′ primer for MLV MA and MA12 was 5′-cggaggctagaaggaaagagATGGGCCAGACTGTTACCACTC-3′ (SEQ ID NO.: 1). The 3′ primer for MA was 5′-cctggaggttctgcactataggATAAAGGGAGGATCGAGGCG-3′ (SEQ ID NO.: 2). The 3′ primer for p12 was 5′-ccctggaggttctgcactataggGAATGCCTGCGAGGTAGTG-3′ (SEQ ID NO.: 3).

Uppercase letters in oligonucleotide sequences represent MLV-derived sequence and lowercase represent HIV-derived sequence. PCR products were cloned into the unique BssHII and SpeI sites in the proviral clone. All PCR generated sequences were confirmed by sequence analysis. Luciferase expressing MLV chimeras were generated by cloning the BssHII and SpeI sites into the identical sites in pNL R-E-Luc (3). The envelope glycoprotein (Env) expression vector for the wild-type (WT) Env was pSHRS HXB Env and the cytoplasmic tail deletion mutant, 147 Env, was pSHRS ε 147 Env (6) The 3T3 XTC cell line was generated by infecting 3T3.T4.CXCR4 cells (5) with helper-free retrovirus expressing the human cyclin T1 gene and selected by selection in puromycin containing medium (DMEM, 10% calf serum, 2: g/ml puromycin). 3T3 XTC cells were confirmed to support HIV-1 Tat function. Human embryonic kidney cells (293) were maintained in DMEM 10% fetal calf serum.

Transfection and infectivity assay. For virus production assays and generation of luciferase expressing viruses 293 cells or 3T3 XTC cells were transfected with Lipofectamine Plus reagent (Invitrogen Life Technologies) according to manufacturer's standard protocol. The virus containing supernatants were harvested 2 days post-transfection. Virus-containing supernatants were cleared of cellular debris by centrifugation at 500 g for 5 min and 0.45 micron filtered. Target cells (HOS T4) were infected with virus supernatants for 2 days at 37° C. The cells then were lysed with 100 ml of luciferase cell culture lysis buffer (Promega). A 20-ml sample of each lysate was assayed for photon emission with a 96-well plate luminometer (LuminoskanRT, Labsystems).

ELISA and western blot analyses. Supernatants were cleared of cellular debris by low speed centrigation 500 g, 5 min, followed by 0.45 micron filtration. Supernatants were inactivated in 0.5% Triton X-100 phosphate buffered saline prior to quantitation by commercial ELISA (NEN Life Sciences). Serial ten-fold dilutions of the supernatants were performed and concentrations determined by comparison to dilutions of the kit-based p24 standards. For western blot analyses, virus particles were purified by centrifugation through a 20% sucrose cushion, 1× TNE buffer (10 mM Tris HCl pH 7.5, 1 mM EDTA, and 100 mM NaCl). Viral pellets were resuspended in 1× SDS Loading buffer and separated by SDS PAGE 4-10% gradient. Proteins were transferred to polyvinyline difluoride (PVDF) membrane (Hybond, P. Amersham Pharmacia Biotech), blocked with 4% nonfat dried milk, 0.1% Tween in PBS. Blots were probed with the following antibodies: human anti HIV pooled patient antiserum (1:5000 dilution). Sheep anti p24 (1:2000 dilution; AIDS Reagent Program), mouse monoclonal anti p17, (1:1000 dilution; AIDS Reagent Program), and Sheep anti HIV-1 gp120 env (1:1000 diluLtion; AIDS Reagent Program). Western blots were developed with horseradish peroxidase-conjugated secondary antibodies (Jackson ImmunoResearch Labs, West Grove, OA), followed by chemiluminescent detection (ECL Amersham).

Deconvolution fluorescence microscopy. Transfected 3T3 TXC cells were plated onto treated microscope slides (VWR brand Superfrost Plus Microslice, VWR). Cells were fixed with 4% paraformaldehyde in PBS for 10 minutes, permeabilized with 0.15% Triton X-100 for 3 minutes, and blocked with PBS containing 3% bovine serum albumin (BSA), 0.2% Tweena dn 0.2% fish gelatin for 1 hr. Primary antibody, a sheep polyclonal antip24, 1:50 dilution (AIDS reagent repository) was applied for 1 hr. at 37 degrees Centigrade. Secondary antibody, Texas Red-conjugated donkey anti-rabbit, 1:100 dilution (Jackson ImmunoResearch Labs), was applied for 1 hr at 37 degrees Centigrade. Slides were directly mounted in DAPI-containing Vectashield (Vector, Burlington, Calif.). The cells were imaged on a Nikon Eclipse 800 fluorescence microscope with 100X oil immersion lens. Image stacks were recorded at 150-nm intervals (z series) with a Hamamatsu Orca CCD camera (Hamamatsu Pliotonics, Bridgewater, N.J.) using the Metamorph imaging system (Universal Imaging Corp, West Chester, Pa.) and de-convolved with Huygens software (Bitplane, Switzerland). The images were then reconstructed in 3-D using Imaris software (Bitplane, Switzerland) for further analysis.

Results

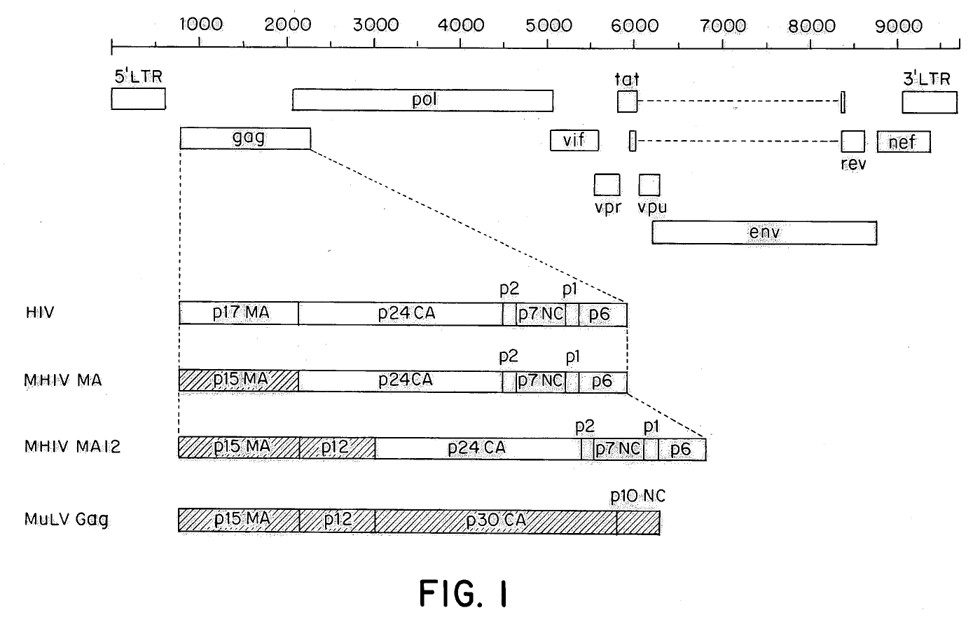

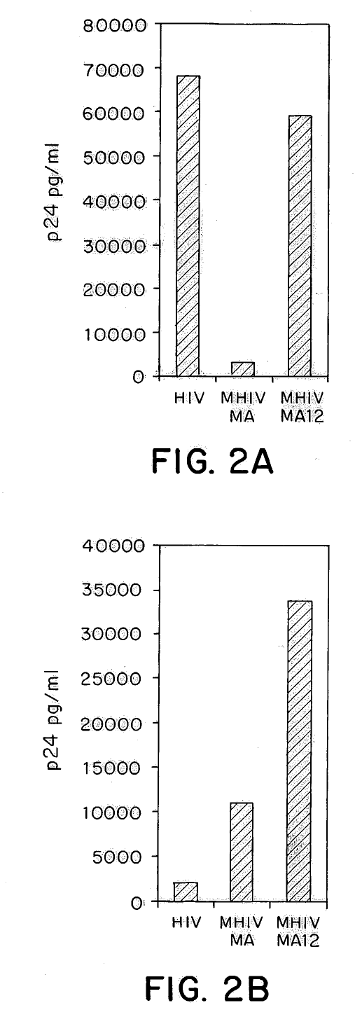

The MA portion of the HIV Gag polyprotein plays a major role in membrane targeting. The phenotype of HIV Gag in mouse cells is one of mislocalization and aggregation and, therefore, the MA portion of HIV Gag was replaced with the MA domain of the murine leukemia virus (FIG. 1). The MLV Gag p12 domain has no equivalent protein in its Gag polypeptide. Because the p12 protein plays important roles in MLV in facilitating Gag processing and maintaining infectivity, Gag chimeras which replace HIV MA with both the MLV MA and p12 domains were made. Both of these Gag chimeras were cloned into the context of full length molecular clones of HIV, pNL4-3, and arc designated MHIV MA for the matrix chimera and MHIV MA12 for the matrix-p12 chimera. Proviral DNAs for the chimeric viruses were transfected into human embryonic kidney 293 cells, and cell supernatants tested for HIV antigen by HIV p24 ELISA. The MA-chimera produced significantly less p24, but the MA-p12 chimera produced levels of HIV p24 antigen comparable to the wild type HIV (FIG. 2A). When the same viral constructs were transfected into NIH 3T3 cells stably expressing human CD4, CXCR4 and Cyclin T1 (3T3 XTC) the two chimeric viruses both produced more supernatant p24 than the wild type HIV (FIG. 2B). In particular, the MHIV MA12 chimera produced fifteen-fold greater levels of supernatant p24 as compared to the wild type HIV.

Western Blot analysis of purified virus particles revealed that the chimeric MHIV-MA12 virus produced a p24 CA protein that was the same size as the wild type HIV p24. The MHIV-MA chimera CA protein was not processed fully into the p24 species and showed instead a major reactive band at 36 KD. This suggests that the HIV protease was able to recognize and cleave the MLV-p12/HIV p24 CA junction to produce a CA protein of the expected size, but did not cleave the MLV-MA/HIV p24 junction efficiently. Western blotting with antibodies against the HIV MA protein confirmed that the HIV MA protein was present only in wild type HIV virus particles and not in either MLV chimera. Because the HIV MA protein is also known to function in envelope recruitment, the virus particles were also tested for their ability to incorporate HIV env. Both MLV-chimeras were found to incorporate significantly less HIV Env than the wild type.

To see if the chimeric Gag proteins were localized to the plasma membrane, deconvolution fluorescence microscopic was performed on transfected 3T3T4X4 Cyclin cells. Transfected cells were stained with polyclonal antibodies against the p24 CA and developed with a Texas Red conjugated secondary antibody. The wild type HIV produced a poorly localized signal that was not localized to the membrane. The MHIV MA12 chimera produced a clear membrane associated, punctate pattern. This pattern resembles the localization pattern previously described for the HIV Gag expressed in human cells (9).

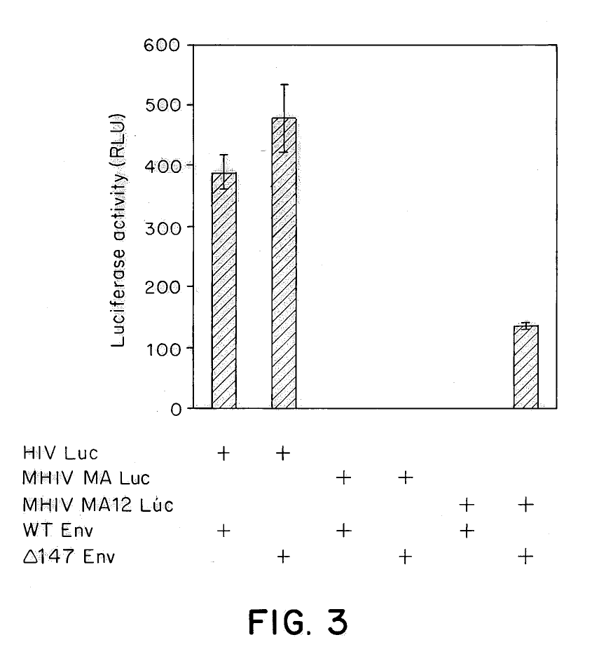

To examine whether the chimeric virus could still produce infectious virus particles the chimeric Gag genes were cloned into a luciferase-expressing HIV molecular clone, pNL R-E-Luc (3.4). These viruses express a highly sensitive enzymatic indicator, firefly luciferase as a measure of the infection efficiency of the HIV. Cotransfection of these Env-deficient viral constructs with an Env-expression vector produces infectious virus. It has previously been reported that MLV does not incorporate HIV Env efficiently. This may be due to steric interference of MLV Gag with the large cytoplasmic tail of HIV Env. However, an HIV Env with a truncated cytoplasmic tail (Env [symbol] 147) is able to pseudotype MLV (16). Therefore, chimeric virus with either the wild type Env or the [symbol] 147 Env were produced. The MHIV MA12 chimera was able to produce infectious particles at 25-30% the level of wild type HIV (FIG. 3). These results suggest that the MHIV MA12 chimera is compatible with other steps in the HIV life cycle. The MHIV-MA chimera did not produce infectious particles with WT or [symbol] 147 env.

Discussion

The MHIV MA12 chimeric Gag overcomes a major impediment to the development of a murine model for HIV infection—the block to assembly of HIV at the plasma membrane. Grafting the MA and p12 domains of MLV Gag into the place of HIV MA resulted in proper membrane localization and a large increase in HIV particle production when transfected into a murine 3T3 cell line. The resulting virus contains a p24 CA protein that is processed appropriately. When produced in human cells, it also maintains the ability of the virus to infect cells efficiently in a single round when provided with an envelope with a truncated cytoplasmic tail. However, because the HIV MA protein plays a critical role in HIV Env incorporation, the chimieric virus fails to carry sufficient Env to retain its infectivity in multiple rounds (data not shown). Additional modifications to the HIV MA12 chimeric virus will be needed to recover its ability to incorporate an HIV Env. Preliminary efforts to test the ability of a chimeric virus carrying the [symbol] 147 env on the viral genome have not resulted in a virus that is able to amplify itself.

The chimera that performs well is one that carries the p12 in addition to MLV MA. The p12 domain has no counterpart in HIV Gag. However, in MLV it is essential for maintaining viral infectivity. The MLV p12 mutant does not process its Gag protein efficiently. It is also defective in the Env processing event that occurs in MLV. The MA-chimeric HIV fails to process p24 fully, suggesting that p12 may in part play a structural role in the Gag polyprotein to allow MLV MA to function. Some have suggested that a polyproline motif in p12 may also serve a function in late assembly analagous to the HIV p6, which lies at the very C-terminus of HIV Gag. It is therefore also possible that part of the gain of function of the MA-p12 chimera may also be due to the addition of another L domain.

The functional replacement of HIV-MA with MLV MA-p12 suggests strongly that it is specifically the HIV MA that fails to function in mouse cells. Previous studies have suggested that the absence of a human-specific factor limits the ability of HIV to assemble in mouse cells. It is now also likely that this human-specific factor acts as an HIV MA co-factor. Biochemical or genetic strategies for identifying human factors that interact with HIV MA may therefore provide an effective means for identifying the human cell specific HIV assembly factor.

Matrix protein is also thought to have a partially redundant function in mediating nuclear import of the preintegration complex during the early phases of infection. This function is thought to play a role in the infection of non-dividing cells. Future experiments will determine whether the matrix chimeric viruses are still able to infect non-dividing cells.

While this invention has been particularly shown and described with reference to preferred embodiments thereof, it will be understood by those skilled in the art that various changes in form and details may be made therein without departing from the spirit and scope of the invention as defined by the appended claims. Those skilled in the art will recognize or be able to ascertain, using no more than routine experimentation, many equivalents to the specific embodiments of the invention described herein. Such equivalents are intended to be encompassed in the scope of the claims.

References:

1. Adachi, A., H. E. Gendelman, S. Koenig, T. Folkcs, R. Willey, A. Rabson, and M. A. Martin. 1986. Production of acquired immunodeficiency syndrome-associated retrovirus in human and nonhuman cells transfected with an infectious molecular clone. J Virol. 59:284-91.

2. Bieniasz, P. D., and B. R. Cullen. 2000. Multiple blocks to human immunodeficiency virus type 1 replication in rodent cells. J Virol. 74:9869-77.

3. Chen, B. K., K. Saksela, R. Andino, and D. Baltimore. 1994. Distinct modes of human immunodeficiency virus type 1 proviral latency revealed by superinfection of nonproductively infected cell lines with recombinant luciferase-encoding viruses. J Virol. 68:654-60.

4. Connor, R. I., B. K. Chen, S. Choe, and N. R. Landau. 1995. Vpr is required for efficient replication of human immunodeficiency virus type-1 in mononuclear phagocytes. Virology. 206:935-44.

5. Deng, H. K., D. Unutmaz, V. N. KewalRamani, and D. R. Littman. 1997. Expression cloning of new receptors used by simian and human immunodeficiency viruses. Nature. 388:296-300.

6. Dubay, J. W., S. J. Roberts, B. H. Hahn, and E. Hunter. 1992. Truncation of the human immunodeficiency virus type 1 transmembrane glycoprotein cytoplasmic domain blocks virus infectivity. J Virol. 66:6616-25.

7. Feng, Y., C. C. Broder, P. E. Kennedy, and E. A. Berger. 1996. HIV-1 entry cofactor: functional cDNA cloning of a seven-transmembrane, G protein-coupled receptor. Science. 272:872-7.

8. Hart, C. E., C. Y. Ou, J. C. Galphin, J. Moore, L. T. Bacheler, J. J. Wasmuth, S. R. Petteway, Jr., and G. Schochetman. 1989. Human chromosome J2 is required for elevated HIV-1 expression in human-hamster hybrid cells. Science. 246:488-91.

9. Hermida-Matsumoto. L., and M. D. Resh. 2000. Localization of human immunodeficiency virus type 1 Gag and Env at the plasma membrane by confocal imaging. J Virol. 74:8670-9.

10. Maddon, P. J., A. G. Dalgleish, J. S. McDougal, P. R Clapham, R. A. Weiss, and R. Axel. 1986. The T4 gene encodes the AIDS virus receptor and is expressed in the immune system and the brain. Cell. 47:333-48.

11. Mariani. R., B. A. Rasala, C. Rutter, K. Wiegers, S. M. Brandt, H. G. Krausslich, and N. R. Landau. 2001. Mouse-human heterokaryons support efficient human immunodeficiency Virus type 1 assembly. J Virol. 75:3141-51.

12. Mariani. R,, G. Rutter, M. E. Harris, T. J. Hope, H. G Krausslich, and N. R. Landau. 2000. A block to human immunodeficiency virus type 1 assembly in murine cells. J ViroL. 74:3859-70.

13. Naviaux. R. K., E. Costanzi, M. Haas, and I. M. Verma. 1996. The pCL vector system: rapid production or helper-free, hih-titer, recombinant retroviruses. J Virol. 70:5701-5.

14. Newstein. M., E. J. Stanbridge, G. Casey, and P. R. Shank. 1990. Human chromosome 12 encodes a species-specific factor which increases human immunodeficiency virus type 1 tat-mediated trans activation in rodent cells. J Virol. 64:4565-7.

15. Ono, A., J. M. Orenstein, and E. O. Freed. 2000. Role of the Gag matrix domain in targeting human immunodeficiency virus type 1 assembly. J Virol. 74:2855-66.

16. Schnierle. B. S., J. Stitz, V. Bosch, F. Nocken, H. Merget-Millitzer, M. Engelstadter, R. Kurth, B. Groner, and K. Cichutek. 1997. Pseudotyping of murine leukemia virus with the envelope glycoproteins of HIV generates a retroviral vector with specificity of infection for CD4-expressing cells. Proc Natl Acad Sci U S A. 94:8640-5.

17. Wei, P., M. E. Garber, S. M. Fang, W. H. Fischer, and K. A. Jones. 1998. A novel CDK9-associated C-type cyclin interacts directly with HIV-1 Tat and mediates its high-affinity, loop-specific binding to TAR RNA. Cell. 92:451-62.

While this invention has been particularly shown and described with references preferred embodiments thereof, it will be understood by those skilled in the art that various changes in form and details may be made therein without departing from the scope of the invention encompassed by the appended claims.

What is claimed is:

1. A functional HIV-MLV Gag chimera, comprising HIV Gag protein and a Gag protein of non HIV origin, which overcomes the block to HIV assembly in non human cells.

2. The functional chimera of claim 1, wherein the Gag protein of non HIV origin is a murine virus Gag protein.

3. The functional chimera of claim 2, wherein the murine virus is Moloney Murine Leukemia Virus.

4. The functional chimera of claim 1, wherein the Gag protein of non HIV origin is Moloney Murine Leukemia Virus Gag matrix protein.

5. The functional chimera of claim 4, wherein the functional chimera additionally comprises Moloney Murine Leukemia Virus p12 protein.

6. The functional chimera of claim 5, additionally comprising HIV Env in which the cytoplasmic tail is truncated in such a manner that the functional chimera produces infectious particles in mouse cells.

7. The functional chimera of claim 6, which produces infectious particles in NIH 3T3 cells that stably express human CD4, human CXCR4 and human Cyclin T1.

8. A chimeric HIV particle that is assembled in non human cells, wherein the chimeric HIV particle comprises chimeric HIV Gag protein in which the HIV Gag matrix domain is replaced by the Gag matrix domain of a murine virus.

9. The chimeric HIV particle of claim 8, wherein the murine virus is the Moloney Murine Leukemia Virus and the non human cells are mouse cells.

10. The chimeric HIV particle of claim 9, additionally comprising HIV Env in which the cytoplasmic tail is truncated in such a manner that the functional chimera produces infectious particles in mouse cells.

11. The functional chimera of claim 10 that produces infectious particles in NIH 3T3 cells that stably express human CD4, human CXCR4 and human Cyclin T1.

12. A chimeric HIV particle that is assembled in non human cells, wherein the chimeric HIV particle comprises chimeric HIV Gag protein in which the HIV Gag matrix domain is replaced by the Gag matrix domain and p12 domain of a murine virus.

13. The chimeric HIV particle of claim 12, wherein the murine virus is the Moloney Murine Leukemia Virus and the non human cells are mouse cells.

14. The chimeric HIV particle of claim 13, additionally comprising HIV Env in which the cytoplasmic tail is truncated in such a manner that the functional chimera produces infectious particles in mouse cells.

15. The functional chimera of claim 14 that produces infectious particles in NIH 3T3 cells that stably express human CD4, human CXCR4 and human Cyclin T1.

16. A nucleic acid construct encoding a chimeric HIV particle that is assembled in non human cells, wherein the chimeric HIV particle comprises chimeric HIV Gag protein in which the HIV Gag matrix domain is replaced by the Gag matrix domain of a murine virus.

17. The nucleic acid construct of claim 16 which is a DNA construct.

18. The DNA construct of claim 17, wherein the murine virus is the Moloney Murine Leukemia Virus and the non human cells are mouse cells.

19. The DNA construct of claim 18, additionally comprising DNA encoding HIV Env in which the cytoplasmic tail is truncated in such a manner that the functional chimera produces infectious particles in mouse cells.

20. A nucleic acid construct encoding a chimeric HIV particle that is assembled in non human cells, wherein the chimeric HIV particle comprises chimeric HIV Gag protein in which the HIV Gag matrix domain is replaced by the Gag matrix domain and p12 domain of a murine virus.

21. The nucleic acid construct of claim 20 which is a DNA construct.

22. The DNA construct of claim 21, wherein the murine virus is the Moloney Murine Leukemia Virus and the non human cells are mouse cells.

23. The DNA construct of claim 22, additionally comprising DNA encoding HIV Env in which the cytoplasmic tail is truncated in such a manner that the chimeric HIV particle produces infectious particles in mouse cells.

24. Mouse cells containing a nucleic acid construct that encodes a chimeric HIV particle that is assembled in mouse cells.

25. Mouse cells of claim 24, wherein the chimeric HIV particle that is assembled in mouse cells comprises chimeric HIV Gag protein in which the HIV Gag matrix domain is replaced by the Gag matrix domain of a murine virus.

26. Mouse cells of claim 25, wherein the murine virus is Moloney Murine Leukemia Virus.

27. Mouse cells of claim 25, wherein the chimeric HIV particle that is assembled in mouse cells additionally comprises HIV Env in which the cytoplasmic tail is truncated in such a manner that the chimeric HIV particle produces infectious particles in mouse cells.

28. Mouse cells of claim 26, wherein the cells express (a) human CD4 and (b) human CCR5, human CXCR4 or both human CCR5 and human CXCR4.

29. Mouse cells of claim 28 which additionally express human Cyclin T1.

30. Mouse cells of claim 24, wherein the chimeric HIV particle that is assembled in mouse cells comprises chimeric HIV Gag protein in which the HIV Gag matrix domain is replaced by the Gag matrix domain and p12 domain of a murine virus.

31. Mouse cells of claim 30, wherein the murine virus is Moloney Murine Leukemia Virus.

32. Mouse cells of claim 31, wherein the chimeric HIV particle that is assembled in mouse cells additionally comprises HIV Env in which the cytoplasmic tail is truncated in such a manner that the chimeric HIV particle produces infectious particles in mouse cells.

33. Mouse cells of claim 32, wherein the cells express (a) human CD4 and (b) human CCR5, human CXCR4 or both human CCR5 and human CXCR4.

34. Mouse cells of claim 33 which additionally express human Cyclin T1.

35. A transgenic nonhuman mammal whose cells express human CD4 and human CCR5, human CXCR4 or both human CCR5 and human CXCR4.

36. The transgenic nonhuman mammal of claim 35, which is a transgenic mouse.

37. The transgenic mouse of claim 36, whose cells contain a nucleic acid construct encoding a chimeric HIV particle that is assembled in cells of the transgenic mouse, wherein the chimeric HIV particle comprises chimeric HIV Gag protein in which the HIV Gag matrix domain is replaced by the Gag matrix domain of a murine virus.

38. The transgenic mouse of claim 37, wherein the nucleic acid construct is a DNA construct and the murine virus is Maloney Murine Leukemia Virus.

39. The transgenic mouse of claim 37, wherein, the chimeric HIV particle that is assembled in mouse cells additionally comprises HIV Env in which the cytoplasmic tail is truncated in such a manner that the chimeric HIV particle produces infectious particles in mouse cells.

40. The transgenic mouse of claim 36 whose cells contain a nucleic acid construct encoding a chimeric HIV particle that is assembled in cells of the transgenic mouse, wherein the chimeric HIV particle comprises chimeric HIV Gag protein in which the HIV Gag matrix domain is replaced by the Gag matrix domain and p12 domain of a murine virus.

41. The transgenic mouse of claim 40, wherein the nucleic acid construct is a DNA construct and the murine virus is Maloney Murine Leukemia Virus.

42. The transgenic mouse of claim 41, wherein, the chimeric HIV particle that is assembled in mouse cells additionally comprises HIV Env in which the cytoplasmic tail is truncated in such a manner that the chimeric HIV particle produces infectious particles in mouse cells.


SEQUENCE LISTING (3 sequences)

SEQ ID NO: 1 - DNA, 42 bp, Unknown
  5′ Primer for MLV MA and MA12
cggaggctagaaggagagagatgggccagactgttaccactc

SEQ ID NO: 2 - DNA, 42 bp, Unknown
  3′ Primer for MA
cctggaggttctgcactataggataaagggaggatcgaggcg

SEQ ID NO: 3 - DNA, 42 bp, Unknown
  3′ Primer for p12
ccctggaggttctgcactatagggaatgcctgcgaggtagtg